(19) United States
(12) Patent Application Publication
(10) Pub. No.: US 2008/0171671 A1
(43) Pub. Date: Jul. 17, 2008

(54) Use of Ethoxylated Amidoamines as Emulsifiers in Drilling Fluids

(76) Inventors: Heinz Mueller, Monheim (DE); Jens Hartmann, Bidnija (DE); Stephan Von Tapavicza, Erkrath (DE); Diana Maeker, Monheim (DE)

Correspondence Address: COGNIS CORPORATION;PATENT DEPARTMENT, 300 BROOKSIDE AVENUE, AMBLER, PA, 19002, US

(21) Appl. No.: 10/595,083

(22) PCT Filed: Oct. 14, 2005
(86) PCT No.: PCT/EP2005/011071

§ 371 Date: Mar. 3, 2008

(30) Foreign Application Priority Data
Oct. 21, 2004 (DE) 10 2004 051 280.9

(51) Int. Cl. C09K 8/04; C07C 229/02

(52) U.S. Cl. 507/218; 560/169

(57) ABSTRACT

The invention relates to the use of ethoxylated derivatives of amidoamines corresponding to general formula (I):

R¹—OC—NR²—[(CH₂)ₙ—NR³]ₘ—CO—R⁴ (I)

In which R¹, R², R³ and R⁴ independently of one another represent a hydrogen atom, a branched or unbranched alkyl or alkenyl group containing 5 to 23 carbon atoms or a CO—CH=CH—COOH group and n is a number of 1 to 6 and m is a number of 1 to 8,

as an emulsifier in drilling fluids which contain at least one continuous oil phase, an aqueous phase and typical additives.

This invention relates to emulsifiers for well servicing compositions and to drilling fluid systems containing the emulsifiers.

It is known that drilling fluids for sinking wells in rock and bringing up the rock cuttings are flowable water- or oil-based systems that are thickened to a limited extent. Oil-based systems are acquiring increasing significance in practice and are used in particular in offshore drilling operations. Oil-based drilling fluids are generally used as so-called invert emulsion muds which consist of a 3-phase system: oil, water and fine-particle solids. Drilling fluids such as these are preparations of the w/o emulsion type, i.e. the aqueous phase is heterogeneously and finely dispersed in the continuous oil phase. A range of additives may be used for stabilizing the system as a whole and for establishing the required performance properties, including in particular emulsifiers and emulsifier systems, weighting agents, fluid loss additives, viscosity adjusters and optionally an alkali reserve.

A key criterion for evaluating the usefulness of invert drilling fluids in practice are their rheological characteristics. Certain viscosity values have to be maintained in drilling fluid systems suitable for practical application. In particular, uncontrolled thickening and hence increases in the viscosity of the drilling fluid have to be strictly prevented because, otherwise, the pipe can become stuck during drilling and can only be freed by expensive, time-consuming measures. In practice, therefore, suitable diluents are added to the drilling fluid systems before and also during drilling. It is known that anionic surfactants from the group of fatty alcohol sulfates, fatty alcohol ether sulfates and alky benzenesulfonates are preferably used for this purpose. In addition, it is important to ensure that the drilling fluid which is pumped into the ground is heated, for example, to temperatures of 150 to 250° F. (66 or 121° C.), depending on the depth, and—in the case of very deep wells—to temperatures of up to 350° F. (178° C.), although it is not always desirable for the rheology of the drilling fluid at high temperatures to be influenced at the same time. Instead, only selective influencing in the critical low temperature range is desirable in many cases. In addition, all additives and auxiliaries used in offshore and onshore drilling fluid systems are expected to satisfy stringent biodegradability and toxicity requirements. Also, the ambient conditions prevailing during drilling operations, such as high temperatures, high pressures, changes in pH caused by the inrush of acidic gases, etc., impose high demands on the choice of possible components and additives. If water-based drilling fluids are used in emulsion form, as is often the case nowadays, the use of emulsifiers is essential.

The selection of emulsifiers for well servicing systems and, more particularly, drilling fluids is primarily aimed at finding substances which lead in maximum stability of the emulsion, even under the extreme conditions of practical application, i.e. an increase in the viscosity of the drilling fluid, more particularly breaking of the emulsion, should be strictly prevented. This applies in particular to emulsions of the water-in-oil type. Several compounds suitable for this purpose are known to the expert, so-called amidoamines in particular playing an important role. Amidoamines based on dimer fatty acids are the subject of EP 0 229 912 A1 which also discloses their use in drilling fluids.

However, there is a constant demand for other suitable emulsifiers for this technical field with particular emphasis on environmental aspects—in this case the ecological compatibility and biodegradability of the substances.

It has been found that certain derivatives of the amidoamines meet these requirements. Accordingly, the present invention relates to the use of ethoxylated derivatives of amidoamines corresponding to general formula (I):

R¹—OC—NR²—[(CH₂)ₙ—NR³]ₘ—CO—R⁴ (I)

in which R¹, R², R³ and R⁴ independently of one another represent a hydrogen atom, a branched or unbranched alkyl or alkenyl group containing 5 to 23 carbon atoms or a CO—CH=CH—COOH group and n is a number of 1 to 6 and m is a number of 1 to 8,
as an emulsifier in drilling fluids which contain at least one continuous oil phase, an aqueous phase and typical additives.

The ethoxylated amidoamines, like the compounds of formula (I) themselves, are already known. To produce the ethoxylated compounds according to the invention, amidoamines corresponding to formula (I) may be reacted with gaseous ethylene oxide at elevated temperature in the presence of basic catalysts, preferably potassium hydroxide or sodium methylate. The reaction temperatures should be in the range from 100 to 150° C. and preferably in the range from 110 to 140° C.

The compounds of formula (II) are also known. They may be prepared by reacting oligoamines or polyamines, preferably diethylene triamine, triethylene tetramine and/or tetraethylene pentamine, and mixtures of these amines with fatty acids. Suitable fatty acids contain between 6 and 24 carbon atoms and may be branched, unbranched, saturated or unsaturated. Tall oil fatty acids, oleic acid, linolenic acid and/or linoleic acid are preferably used. Mixtures of the acids may also be used. In addition, it can be of advantage to use dicarboxylic acid and preferably maleic acid, for example in the form of its anhydride, during the reaction of the fatty acids with the amines.

A preferred compound of formula (I) corresponds to the following general formula:

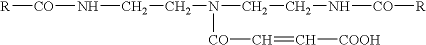

where R stands for tall oil fatty acid alkyl groups. Tall oil itself is a mixture of fatty acids, resin acids, so-called oxy-acids (and other oxidized resin and fatty acids) and other unsaponifiable components. Its composition varies widely according to the nature of the processed wood and its geographic origin: 15-55% fatty acids, ca. 20-65% resin acids, 1-8% oxy-acids and 6-30% other unsaponifiable components for an acid value of ca. 90-160. Tall oil is fractionated by distillation into first runnings and tall oil pitch, which are mainly burnt, and into fatty acids and tall oil resin. At least 97% of the tall oil fatty acids (1^{st} quality) or 67% (resin acid content of 25-30%) consist of fatty acids (% by weight, based on the total quantity of fatty acids in brackets): linoleic acid and conjugated C₁₈ fatty acids (45-65), oleic acid (25-45), 5,9,12-octadecatrieenoic acid (5-12) and saturated fatty acids (1-3).

Ethoxylated amidoamines which have proved to be advantageous for the purposes of the present technical teaching are those of which the content of ethylene oxide groups is between 1 and 10 parts per part amidoamine, the range from 1 to 7 being preferred and the range from 1 to 5 being particularly preferred. Ethoxylated derivatives of amidoamines corresponding to formula (I), where R₁ and R⁴ represent C₅₋₂₃ alkyl and/or alkenyl groups and R³ is a CO—CH=CH—COOH group and/or a hydrogen atom are also preferred.

The ethoxylated amidoamines according to the invention are preferably used as emulsifiers in drilling fluid systems which, based on the liquid phase as a whole, contain 10 to 30% by weight water and hence 90 to 70% by weight of the oil phase. To this end, the ethoxylated amidoamines are used in quantities of 0.1 to 25% by weight, preferably in quantities of 0.1 to 10% by weight and more particularly in quantities of 0.1 to 5% by weight, based on the weight of the drilling fluid as a whole.

The use of the ethoxylated amidoamines according to the invention results in an improved filtrate value of the particular drilling fluid in relation to standard amidoamine-based emulsifiers. In addition, the drilling fluids formulated with the ethoxylated emulsifiers have good rheology values.

The present invention also relates to well servicing compositions, more particularly drilling fluids, flowable and pumpable at 5 to 20° C. which are based on a continuous oil phase, optionally in admixture with a limited quantity of a disperse aqueous phase, (w/o invert type), and which if desired contain dissolved and/or dispersed standard auxiliaries, such as thickeners, emulsifiers, fluid loss additives, wetting agents, fine-particle weighting agents, salts, alkali reserves and/or biocides, characterized in that, in their oil phase, they contain compounds selected from the following classes:
(a) carboxylic acid esters corresponding to formula (II):

R′—COO—R″ (II)

where R′ is a saturated or unsaturated, linear or branched C₅₋₂₃ alkyl group and R″ is a C₁₋₂₂ alkyl group which may be saturated or unsaturated, linear or branched,
(b) linear or branched C₈₋₃₀ olefins,
(c) water-insoluble, symmetrical or nonsymmetrical ethers of monohydric alcohols of natural or synthetic origin which may contain 1 to 24 carbon atoms,
(d) water insoluble alcohols corresponding to formula (III):

R′″—OH (III)
where R′″ is a saturated, unsaturated, linear or branched C₈₋₂₄ alkyl group,
(e) carbonic acid diesters,
(f) paraffins,
(g) acetals.

These compositions contain oils belonging to the above-mentioned groups either on their own or in the form of mixtures with one another in the continuous oil phase. In a particularly preferred embodiment, the oil phase is formed by the carboxylic acid esters of formula (II), more particularly those described in EP 0 374 672 or EP 0 386 636. In another particularly preferred embodiment of the teaching according to the invention, the ethoxylated compounds based on amidoamines formula (I) compounds of formula (I) are used in invert drilling fluid emulsions of which the oil phase contains esters of formula (II), in which R′ is an alkyl group containing 5 to 21 carbon atoms, preferably 5 to 17 carbon atoms and more particularly 11 to 17 carbon atoms. Particularly suitable alcohols in such esters are based on branched or unbranched alcohols containing 1 to 8 carbon atoms, for example on methanol, isopropanol, isobutanol or 2-ethylhexanol.

Alcohols containing 12 to 18 carbon atoms are also preferred. Particularly preferred esters are saturated C₁₂₋₁₄ fatty acid esters or unsaturated C₁₆₋₁₈ fatty acids with isopropyl, isobutyl or 2-ethylhexanol as the alcohol component. 2-Ethylhexyl octanoate is also suitable. Other suitable esters are acetic acid esters, particularly acetates of C₈₋₁₈ fatty alcohols. Oil phases such as these—normally called carrier fluids—are known, for example, from earlier Cognis patents/patent applications, cf. in particular European patent applications EP 0 374 671, EP 0 374 672, EP 0 382 070, EP 0 386 638. Oil phases based on linear olefins are also known to the expert, cf. EP-A-0 765 368. Branched esters (a) as disclosed, for example in WO 99/33932 (Chevron) or in BP 0 642 561 (Exxon) are also suitable carrier fluids in the process according to the invention; the esters disclosed therein are part of the disclosure of the present invention. Mixtures of these preferred esters with one another are also preferred. In another preferred embodiment, the oil phase contains α-olefins or internal olefins (IOs) or poly-α-olefins (PAOs) as component (b). The IOs or IO mixtures present in the oil phase according to the invention then contain corresponding compounds with 12 to 30 carbon atoms in the molecule, preferably 14 to 24 carbon atoms and more particularly up to 20 carbon atoms in the molecule. If α-olefins are present as the oil phase, α-olefins based on fatty acids containing 12 to 18 carbon atoms are preferably used, saturated α-olefins being particularly preferred. These preferred mixtures are the subject of applicants' EP 0 765 368 A1. Other suitable constituents of the oil phase are water-insoluble symmetrical or nonsymmetrical ethers (c) of monohydric alcohols of natural or synthetic origin, the alcohols containing from 1 to 24 carbon atoms. Corresponding drilling fluids are the subject of European patent application EP 0 472 557. Water-soluble alcohols of group (d) can also be preferred constituents of the oil phase in the context of the present technical teaching. In addition, carbonic acid diesters (e) according to European patent application EP 0 532 570 can be suitable constituents of the oil phase. These compounds can make up the oil phase as a whole or parts thereof. Paraffins (f) and/or acetals (g) may also be used as constituents of the oil phase. Mixtures of compounds (a) to (g) with one another may also be used. In a preferred embodiment, at least 50% by weight of the oil phase of the emulsions according to the invention consists of such preferred compounds (a) to (g), systems in which 60 to 80% and more particularly 100% by weight of the oil phase consists of compounds (a) to (g) or mixtures thereof being particularly preferred. The oil phases themselves then preferably have flash points above 85° C. and preferably above 100° C. They are formulated in particular as invert drilling fluids of the w/o type and preferably contain the disperse aqueous phase in quantities of about 5 to 30% by weight. The continuous oil phases of such drilling fluids according to the invention have a Brookfield (RVT) viscosity at 0 to 5° C. of below 50 mPa·s and preferably not above 40 mPa·s. The pH of the fluids is preferably adjusted to a value in the range from about neutral to moderately basic and, more particularly, to a value in the range from about 7.5 to 11; the use of lime as an alkali reserve can be particularly preferred. Water is also a constituent of the described drilling fluids. The water is preferably present in the invert emulsions in quantities of at least about 0.5% by weight. In a preferred embodiment, however, the invest emulsions contain at least 5 to 10% by weight of water. The water in drilling fluid systems of the type described herein always contains quantifies of electrolytes to equalize the osmotic gradient between the drilling fluid and the formation water, calcium and/or sodium salts representing the preferred electrolytes. CaCl₂ in particular is commonly used. However, other salts from the group of alkali metals and/or alkaline earth metals, for example potassium acetates and/or formates, are also suitable.

Other preferred mixing ratios are 80% by weight oil phase to 20% by weight water phase. The drilling fluids according to the invention may also contain other typical additives and auxiliaries such as, in particular, other emulsifiers, weighting agents, fluid loss additives, thickeners and alkali reserves, more particularly lime (=Ca(OH)₂), and also biocides and so-called wetting agents which improve the wettability of surfaces.

Emulsifiers suitable for use in practice are systems which are suitable for forming the required w/o emulsions. Selected oleophilic fatty acid salts, for example those based on amidoamine compounds, are particularly suitable. Emulsifiers of the type in question here are marketed as highly concentrated active-component preparations and may be used, for example, in quantities of about 2.5 to 5% by weight and, more particularly, in quantities of ca. 3 to 4% by weight, based on oil phase.

In practice, hydrophobicized lignite in particular is used as a fluid loss additive and, hence, in particular to form a dense coating in the form of a substantially liquid-impermeable film on the walls of the well. Suitable quantities are, for example, about 5 to 20 lb/bbl, preferably 5 to 10 lb/bbl and more particularly 5 to 8% by weight, based on the oil phase.

In drilling fluids of the type in question here, the thickener normally used is a cationically modified fine-particle bentonite which may be used in particular in quantifies of ca. 8 to 10 and preferably 2 to 5 lb/bbl or in the range from 1 to 4% by weight, based on oil phase. The weighting agent normally used to establish the necessary pressure equilibrium is barite (BaSO₄), of which the quantities added are adapted to the particular conditions to be expected in the well. For example, the specific of gravity of the drilling fluid can be increased by addition of barite to values of up to about 2.5 and preferably in the range from ca. 1.3 to 1.6. Another suitable weighting agent is calcium-carbonate.

The use of the emulsifiers according to the invention based on ethoxylated amidoamines also has advantages from the ecological perspective. These emulsifiers surprisingly show both a low toxicity towards marine microorganisms and also good biodegradability. Products of the type in question are supposed to have a value of ca. 20% in OECD Test 306; the products according to the invention have values of 40% or better. The preferred emulsifiers with degrees of ethoxylation of 1 to 10, preferably 1 to 7 and more particularly 1 to 5 show the best results in both categories.

### EXAMPLES

Preparation of the Amidoamine: In an autoclave, 1 mol of a tall oil fatty acid amidoamine based on triethylene triamine was heated to 140° C. with 5 mol ethylene oxide in the presence of NaOCH₃ as catalyst. The reaction product had the following characteristics: acid value (to DIN EN ISO 3682 QC 1313.1); <10; amine value (determined to Houben-Weyl QC 1321.0); <10.

### Performance Tests

### Example 1

In order to test the properties of the amidoamines according to the invention, various invert drilling fluids of the water-in-oil (w/o) type were formulated in the presence of typical amidoamine emulsifiers and the ethoxylated compounds according to the invention. To this end, invert emulsions with the following general composite were tested:

| | | |
|---|---|---|
| oil phase¹⁾ | 136 | ml |
| water | 77 | ml |
| thickener²⁾ | 2 | g |
| emulsifier | x | g |
| Ca(OH)₂ | 2 | g |
| fluid loss additive | 7 | g |
| barium sulfate | 327 | g |
| CaCl₂•2H₂O | 27 | g |
| oil-to-water ratio | 70:30 | (v/v) |
| density | 14 | lb/gal (1.7 g/l) |

¹⁾C₁₆₋₁₈-α-olefin, isomerized (Chevron); density at 20° C. 0.785 g/cm³, Brookfieid (RVT) viscosity at 20° C. 5.5 mPa · s
²⁾modified organophilic bentonite, Geltons II (Baroid)

These fluids were tested both with a standard amidoamide-based emulsifier (EZ-Mul, Baroid) and with the ethoxylated products according to the invention.

The rheological characteristics of the fluids, namely plastic viscosity (PV), yield point (YP) and gel strength (gels 10 “/”) after 10 sec. and 10 mins., were determined with a Fann SR 12 rheometer (Fann).

Electrical stability was also measured. The drilling fluid was then tested in a roller oven (Baroid) for 16 hours at temperatures of 121° C. (250° F.). In the Tables, AHR=after hot rolling, BHR=before hot rolling. Filtrate values were determined in a high-temperature high-pressure (HTHP) cell.

The results are set out in the following Table.

TABLE 1

| Emulsifier | A | | B | | C | | D | |
|---|---|---|---|---|---|---|---|---|
| Amidoamine | 5.2 g | | | | | | | |
| Amidoamine | | | 5.2 g | | 7.0 g | | 4.0 g | |
| ethoxylate | | | | | | | | |
| (5 EO) | | | | | | | | |
| | BHR | AHR | BHR | AHR | BHR | AHR | BHR | AHR |
| Electrical | 390 | 290 | 320 | 260 | 310 | 270 | 340 | 280 |
| stability V | | | | | | | | |
| PV cP | 27 | 27 | 29 | 26 | 26 | 25 | 26 | 24 |
| YP lb/100 ft² | 19 | 13 | 12 | 12 | 8 | 15 | 14 | 14 |
| Gels 10″/10′ | 8/9 | 7/8 | 6/7 | 6/7 | 5/6 | 6/6 | 7/8 | 7/8 |
| lb/100 ft² | | | | | | | | |

### Example 2

A fluid containing 173 ml of the above-mentioned oil phase was tested in another test. The results are set out in Table 2.

TABLE 2

| Emulsifier | E | | F | | G | |
|---|---|---|---|---|---|---|
| Amidoamine | 8 g | | | | | |
| Amidoamine ethoxylate | | | 8 g | | 4 g | |
| (5 EO) | | | | | | |
| | BHR | AHR | BHR | AHR | BHR | AHR |
| Electricl stability V | 380 | 340 | 380 | 320 | 350 | 310 |
| PV cP | 24 | 27 | 26 | 26 | 26 | 25 |
| YP lb/100 ft² | 17 | 18 | 8 | 9 | 11 | 14 |
| Gels 10″/10′ lb/100 ft² | 7/8 | 8/10 | 4/6 | 5/7 | 5/8 | 7/9 |

It can be seen that the use of the ethoxylated amidoamines, even in a reduced quantity, still leads to drilling fluids with good rheological properties.

### Example 3

In order to assess the influence of the degree of ethoxylation, ethoxylated amidoamines with various percentage contents of ethylene oxide were tested as emulsifiers in the drilling fluid described m Example 2.

TABLE 3

| Emulsifier | H | | I | | J | | K | |
|---|---|---|---|---|---|---|---|---|
| Amidoamine | 8 g | | | | | | | |
| Amidoamine | | | 8 g | | | | | |
| ethoxylate | | | | | | | | |
| (5 EO) | | | | | | | | |
| Amidoamine | | | | | 8 g | | | |
| ethoxylate | | | | | | | | |
| (10 EO) | | | | | | | | |
| Amidoamine | | | | | | | 8 g | |
| ethoxylate | | | | | | | | |
| (15 EO) | | | | | | | | |
| | BHR | AHR | BHR | AHR | BHR | AHR | BHR | AHR |
| Electrical Stability V | 390 | 300 | 360 | 200 | 470 | 200 | 400 | 200 |
| PV cP | 26 | 25 | 27 | 25 | 30 | 27 | 26 | 26 |
| YP lb/100 ft² | 18 | 14 | 13 | 8 | 2 | 5 | 6 | 3 |
| Gels 10″/10′ lb/100 ft² | 8/9 | 6/7 | 6/6 | 4/5 | 4/4 | 3/4 | 5/5 | 4/3 |
| HTHP total ml | | 1.6 | | 1.2 | | 2.0 | | 0.4 |
| HTHP water ml | | 0 | | 0 | | 0 | | 0 |
| HTHP oil ml | | 1.6 | | 1.2 | | 2.0 | | 0.4 |
| HTHP temp. ° F. | | 250 | | 250 | | 250 | | 250 |

It can be seen that amidoamine reacted with 5 parts ethylene oxide imparts favorable rheological properties, products with a relatively high EO degree leading to poorer values, particularly for yield point (YP) and gel strength, in direct comparison with the low-EO product.

### Example 4

A drilling fluid was prepared as in Example 2 using a C₁₁₋₂₁ paraffin oil (PureDrill IA 35; CAS 178603-63-9). The results are set out in Table 4.

TABLE 4

| Emulsifier | L | | M | |
|---|---|---|---|---|
| Amidoamine | 8 g | | | |
| Amidoamine ethoxylate (5 EO) | | | 8 g | |
| | BHR | AHR | BHR | AHR |
| Electrical stability V | 470 | 320 | 340 | 230 |
| PV cP | 33 | 29 | 31 | 29 |
| YP lb/100 ft² | 9 | 10 | 8 | 8 |
| Gels 10″/10′ lb/100 ft² | 5/6 | 5/6 | 4/5 | 5/5 |
| HTHP total ml | | 1.4 | | 3.0 |
| HTHP water ml | | 0 | | 0 |
| HTHP oil ml | | 1.4 | | 3.0 |
| HTHP temp. ° F. | | 250 | | 250 |

### Example 5

The toxicity of the ethoxylated amidoamines according to the invention was determined to ISO 14669: 1999 (E) on Acartia tonsa and to BS EN ISO 10253: 1998 on Skeletonema costatum. The biodegradability of the amidoamines was determined to OECD 306.

The results for an ethoxylated amidoamine according to the invention and for a commercially available non-ethoxylated amidoamine (EZ-Mul®, Baroid) are set out in the following Table.

TABLE 5

| | Acartia | Skeletonema | Biodegradabiity in seawater |
|---|---|---|---|
| | tonsa | costatum | to OECD 306 |
| Amidoamine | 6600 | 54.69 | 41% |
| ethoxylate (5 EO) | | | |
| Amidoamine | >2000 | <10 | 7.5% |

1-10. (canceled)

11. An ethoxylated derivative of an amidoamine according to the general formula (1):
R¹—CO—NR²—[(CH₂)ₙ—NR³]ₘ—CO—R⁴ (1)
in which R₁, R², R³ and R⁴ independently of one another represent a hydrogen atom, a branched or unbranched alkyl or alkenyl group containing 5 to 23 carbon atoms or a CO—CH=CH—COOH group and n is a number of 1 to 6 and m is a number of 1 to 8, as an emulsifier in drilling fluids which contain at least one continuous oil phase, an equeous phase and additives.

12. The derivative according to claim 11, wherein the derivative contains 1 to 10 parts ethylene oxide per part amidoamine according to formula (1).

13. The derivative according to claim 11, wherein the derivative contains 1 to 7 parts ethylene oxide per part amidoamine according to formula (1).

14. The derivative according to claim 11, wherein the derivative contains 1 to 5 parts ethylene oxide per part amidoamine according to formula (1).

15. The derivative according to claim 11, wherein R¹ and R⁴ represent an alkyl and/or alkenyl group containing 5 to 23 carbon atoms and R³ is a CO—CH=CH—COOH group and/or hydrogen atom.

16. The derivative according to claim 11, wherein the compound according to formula (1) is produced by reaction of a tall oil fatty acid with an oligo- or polyethylene amine.

17. The derivative according to claim 16, wherein the polyethylene amine is selected from the group consisting of diethylene triamine, triethylene tetramine, tertaethylene pentamine, and mixtures thereof.

18. The derivative according to claim 11, present as an emulsifier in a drilling fluid in an amount of about 0.1 to 25% by weight of the total weight of drilling fluid.

19. The derivative according to claim 11, present as an emulsifier in a drilling fluid in an amount of about 0.1 to 10% by weight of the total weight of drilling fluid.

20. The derivative according to claim 11, present as an emulsifier in a drilling fluid in an amount of about 0.1 to 5% by weight of the total weight of drilling fluid.

21. The derivative according to claim 11, wherein the drilling fluid is a water-in-oil fluid.

22. The derivative according to claim 11, wherein the drilling fluid further comprises a component selected from the group consisting of a weighting agent, a fluid loss additive, a wetting agent, an alkali reserve, a thickener, a biocide and mixtures thereof.

23. The derivative according to claim 11, wherein the derivative is produced by reaction of amidoamines according to formula (1) with ethylene oxide at temperatures of 100 to 150° C. in the presence of a catalyst selected from the group consisting of potassium hydroxide or sodium methylate.

24. The derivative according to claim 11, wherein the derivative is produced by reaction of amidoamines according to formula (1) with ethylene oxide at temperatures of 110 to 140° C. in the presence of a catalyst selected from the group consisting of potassium hydroxide or sodium methylate.

25. A composition, comprising:
an ethoxylated derivative of an amidoamine according to the general formula (1):
R¹—CO—NR²—[(CH₂)ₙ—NR³]ₘ—CO—R⁴ (1)
in which R¹, R², R³ and R⁴ independently of one another represent a hydrogen atom, a branched or unbranched alkyl or alkenyl group containing 5 to 23 carbon atoms or a CO—CH=CH—COOH group and n is a number of 1 to 6 and m is a number of 1 to 8; and
a continuous oil phase in admixture with a limited quantity of a disperse aqueous phase (w/o invert type).

26. The composition according to claim 25, further comprising a component selected from the group consisting of a weighting agent, a fluid loss additive, a wetting agent, an alkali reserve, a thickener, a biocide and mixtures thereof.

27. The composition according to claim 25, wherein the continuous oil phase is selected from a group consisting of:
(a) carboxylic acid esters corresponding to formula (II): R′—COO—R″ (II)
wherein R′ is saturated or unsaturated, linear or branched C₅₋₂₃ alkyl group and R″ is a C₁₋₂₂ alkyl group which may be saturated or unsaturated, linear or branched;
(b) linear or branched C₈₋₂₀ olefins;
(c) water-insoluble, symmetrical or nonsymmetrical ethers of monohydric alcohols of natural or synthetic origin which may contain 1 to 24 carbon atoms;
(d) water-insoluble alcohols corresponding to formula (III): R′″—OH where R′″ is a saturated, unsaturated, linear or branched C₂₋₂₄ alkyl group;
(e) carbonic acid esters;
(f) paraffins; and
(g) acetals.
